(19) United States
(12) Patent Application Publication
(10) Pub. No.: US 2003/0191140 A1
(43) Pub. Date: Oct. 9, 2003

(54) Polymorphic form of 3-[2-[4-(6-fluoro-1,2-benzisoxazol-3-yl)-1-piperidinyl]ethyl]-6,7,8,9-tetrahydro-2-methyl-4H-pyrido[1,2-alpha]pyrimidin-4-one and formulations thereof

(76) Inventors: Inigo Pfeiffer, Wilmington, NC (US); Robert R. Whittle, Wilmington, NC (US); Grayson Walker Stowell, Wilmington, NC (US); Linda B. Whittall, Wilmington, NC (US)

Correspondence Address: MYERS BIGEL SIBLEY & SAJOVEC, PO BOX 37428, RALEIGH, NC, 27627, US

(21) Appl. No.: 10/108,729

(22) Filed: Mar. 28, 2002

(51) Int. Cl. A61K 31/519; C07D 487/04

(52) U.S. Cl. 514/259.41; 544/282

(57) ABSTRACT

A novel polymorphic form of risperidone (3-[2-[4-(6-fluoro-1,2-benzisoxazol-3-yl)-1-piperidinyl]ethyl]-6,7,8,9-tetrahydro-2-methyl-4H-pyrido[1,2-α]pyrimidin-4-one) is useful in pharmaceutical compositions, either in pure form or in combination with other forms of risperidone.

### BACKGROUND OF THE INVENTION

1. Field of the Invention

The present invention relates to compositions and methods of preparing novel forms of the free base of 3-[2-[4-(6-fluoro-1,2-benzisoxazol-3-yl)-1-piperidinyl]ethyl]-6,7,8,9-tetrahydro-2-methyl-4H-pyrido[1,2-α]pyrimidin-4-one (hereinafter referred to by its common name “risperidone”).

2. Description of Related Art

The compound 3-[2-[4-(6-fluoro-1,2-benzisoxazol-3-yl)-1-piperidinyl]ethyl]-6,7,8,9-tetrahydro-2-methyl-4H-pyrido[1,2-α]pyrimidin-4-one is generally known as the pharmaceutically active compound risperidone. Risperidone is a free base and used as an active pharmaceutical ingredient (API) for use in the preparation of drug products.

Risperidone has the following chemical structure:

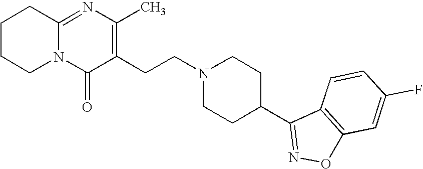

Risperidone is known to be useful in the treatment of psychotic diseases, and has been disclosed in, for example, U.S. Pat. No. 4,804,663 to Kennis et al., which is assigned to Janssen Pharmaceutica N.V. of Beerse, Belgium (see Example 5), the disclosure of which is incorporated herein by reference. Risperidone free base is the API in the pharmaceutical drug product marketed under the trademark RISPERDAL® (Janssen Pharmaceutical Products, LP, Titusville, N.J.).

The existence of various polymorphic forms of risperidone cannot be discerned from the available scientific literature which teaches a single crystalline form (see e.g., “Structure of 3-{2-[4-(6-fluoro-1,2-benzisoxazol-3-yl)-1-piperidinyl]ethyl}-6,7,8,9-tetrahydro-2-methyl-4H-pyrido[1,2-α]pyrimidin-4-one (Risperidone)”, Acta. Cryst. (1993) C 49, 1698-1700). The paper reports the crystal structure of risperidone API performed on a risperidone sample provided by Dr. J. P. Tollenaere, Janssen Pharmaceutica of Beerse, Belgium (hereinafter referred to as the “Reported Risperidone”). This article details the crystal structure and parameters associated with the structural analysis. The structure was reported on the sample provided, but no discussion is provided within the paper as to the nature of the sample analyzed, such as the crystallization or recystallization procedures conducted on the sample. The polymorphic form of risperidone typified by the Reported Risperidone (hereinafter referred to as “Form B” of Risperidone) is the only known crystalline form of risperidone in scientific literature, and the procedures for synthesizing risperidone in U.S. Pat. No. 4,804,663 to Kennis et al. formed Form B risperidone (see, i.e., Example 5).

Polymorphic forms of the same drug substance or API, as administered by itself or formulated as a drug product (also known as the final or finished dosage form), are well known in the pharmaceutical art to affect, for example, the solubility, stability, flowability, tractability, and compressibility of drug substances and the safety and efficacy of drug products (see, e.g., Knapman, K. Modern Drug Discoveries, March, 2000: 53). So critical are the potential effects of different polymorphic forms in a single drug substance on the safety and efficacy of the respective drug product(s) that the United States Food and Drug Administration (FDA) requires each drug substance manufacturer, at least, to control its synthetic processes such that the percentages of the various respective polymorphic forms, when present, must be controlled and consistent among batches and within the drug substance/product's specification as approved by the FDA. Left uncontrolled in synthetic processes, the percentage of a given polymorph outside of an FDA approved specification could render the adulterated batches unfit for commercial sale. Accordingly, the FDA typically requires full characterization of each drug substance used in each drug product marketed in the United States, including the identification and control of polymorphic forms. The FDA further requires robust synthetic process specifications and controls which consistently produce the respective drug substance and drug product.

Unfortunately, the detection of various polymorphic forms of a single drug substance is not always readily discernable by pharmaceutical chemists. Such a drug substance would not be necessarily manufactured with appropriate controls, potentially leaving the attendant safety and efficacy risks unaddressed.

### SUMMARY OF THE INVENTION

A novel crystalline form of risperidone, Form A, which has now been prepared and characterized, is clearly distinguishable from other polymorphic forms of risperidone by X-ray powder diffraction and other methods of solid-state characterization. In accordance with the present invention, Form A of risperidone, can be obtained in a pure form or in combination with other polymorphic forms of risperidone. Form A is stable, and can be prepared free from contamination by solvates such as water or organic solvents including, for example, acetonitrile. As such, Form A is also useful for the commercial preparation of pharmaceutical formulations such as tablets and capsules.

Accordingly, it is an object of the present invention to provide novel compositions, pharmaceutical formulations, and methods of using the novel polymorphic forms of the present invention, and combinations thereof.

The present invention provides a novel polymorphic form of risperidone, either in pure or substantially pure form or as combinations of the novel form with other polymorphic forms of risperidone, each of which can be useful for providing enhanced biological, handling and/or manufacturing characteristics, particularly when prepared in pharmaceutical dosage forms.

### BRIEF DESCRIPTION OF THE DRAWINGS

FIG. 1 shows an ORTEP drawing of the contents of the asymmetric unit of Form A risperidone;

FIG. 2 shows an ORTEP drawing of the contents of the asymmetric unit of Form B risperidone;

FIG. 3 shows an ORTEP drawing comparison of two unique molecules in the single crystal structure of Form A risperidone to Form B risperidone viewed along the C12-C8-C9 plane;

FIG. 4 shows an ORTEP drawing comparison of two unique molecules in the single crystal structure of Form A risperidone to Form B risperidone viewed along the C9-C8-C12 plane;

FIG. 5 shows an ORTEP drawing comparison of two unique molecules in the single crystal structure of Form A risperidone to Form B risperidone viewed along the C23-C22-C21 plane;

FIG. 6 illustrates a Differential Scanning Calorimetry (DSC) thermogram for Form A risperidone;

FIG. 7 illustrates a DSC thermogram for Form B risperidone;

FIG. 8 illustrates a DSC thermogram for the combination of Forms A and B risperidone;

FIG. 9 illustrates an X-ray powder diffraction (XRD) pattern for Form A risperidone;

FIG. 10 illustrates an XRD pattern for Form B risperidone;

FIG. 11 illustrates an XRD pattern comparing Form A risperidone and Form B risperidone;

FIG. 12 illustrates an XRD pattern for the mixture of Form A risperidone and Form B risperidone; and,

FIG. 13 illustrates a High Performance Liquid Chromatography (HPLC) chromatographic overlay comparing Form A and Form B of risperidone.

### DETAILED DESCRIPTION OF THE INVENTION

It has been discovered that risperidone drug substance, generally used to prepare RISPERDAL® and potential generic drugs thereto (risperidone API), has not been fully investigated and characterized as only one particular crystalline form of risperidone (Form B) has previously been reported. It has been unexpectedly discovered that risperidone API drug substance may be prepared so that novel Form A is present by itself or in combination with other forms of risperidone. The two identified polymorphs, novel Form A and previously identified Form B, are correlated to the relative melting point of each polymorph in risperidone, from lowest to highest. Novel Form A also may be combined with other forms of risperidone, such as amorphous risperidone (i.e., risperidone without any particular crystalline form).

Preparation of risperidone, 3-[2-[4-(6-fluoro-1,2-benzisoxazol-3-yl)-1-piperidinyl]ethyl]-6,7,8,9-tetrahydro-2-methyl-4H-pyrido[1,2-α]pyrimidin-4-one, is described in U.S. Pat. No. 4,804,663. The compositions of the present invention are preferably prepared by using such risperidone API as the starting material in the processes used to prepare the API compositions of the present invention. The recrystallization and heating methods set forth below can be used as the final steps in many crystallization processes for the preparation of risperidone. Preferred methods for the preparation of Form A are set forth below, but are not intended to limit the scope of the present invention. The present invention is directed to polymorphic Form A of risperidone, and combinations thereof with other polymorphic forms, and mixtures therewith, the preparation thereof, pharmaceutical formulations thereof, and the use of such polymorphs, preferably in pharmaceutical formulations, for the therapeutic treatment of subjects in need of treatment. The polymorphic forms of the present invention were characterized using single crystal X-ray crystallography (XRC), differential scanning calorimetry (DSC), X-ray powder diffraction (XRD) and High Performance Liquid Chromatography (HPLC) analysis as discussed below. Characterization with some of these methods reveals distinctive features for each particular polymorphic form. For example, pure Form A provides a distinct range of significant peaks when analyzed by XRD. These significant peaks will be present with XRD analysis for pure Form A as well as for samples containing Form A in combination with other polymorphic forms of risperidone.

It has further been discovered that Form A can be prepared in pure or substantially pure polymorphic form in robust, controllable, synthetic processes. Substantially pure Form A is defined by an amount of Form A wherein the amount of risperidone polymorphs other than Form A does not exceed an amount greater than about ten percent (w/w) and, preferably, does not exceed an amount greater than about five percent (w/w).

For the purpose of this invention, the term “pure” refers to Form A of risperidone being in a concentration such that other risperidone polymorphs are present in amounts generally below limits detectable by conventional technology, as taught herein. Although the present invention provides for pure and substantially pure Form A of risperidone, it is particularly preferred to control the ratio of all polymorphic forms to provide a consistent pharmaceutical API.

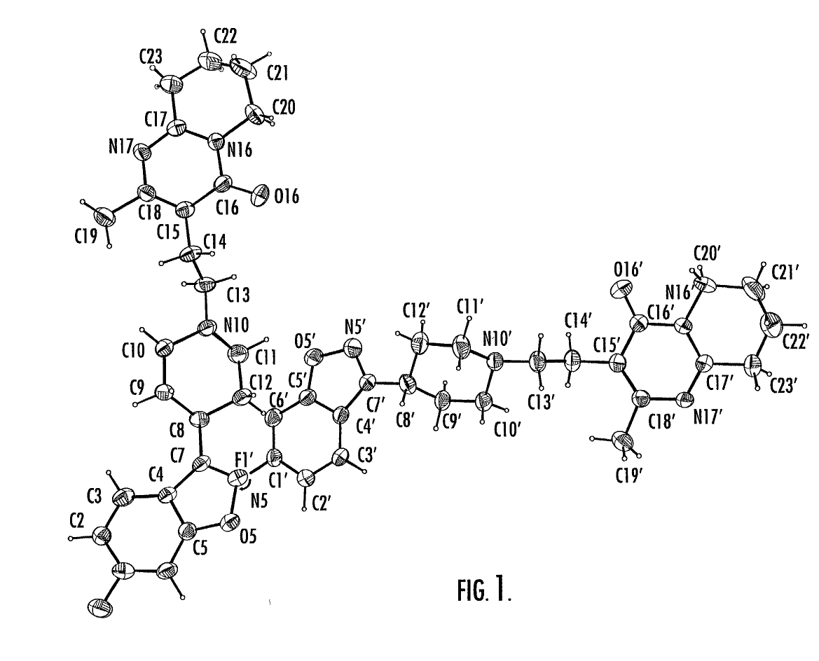

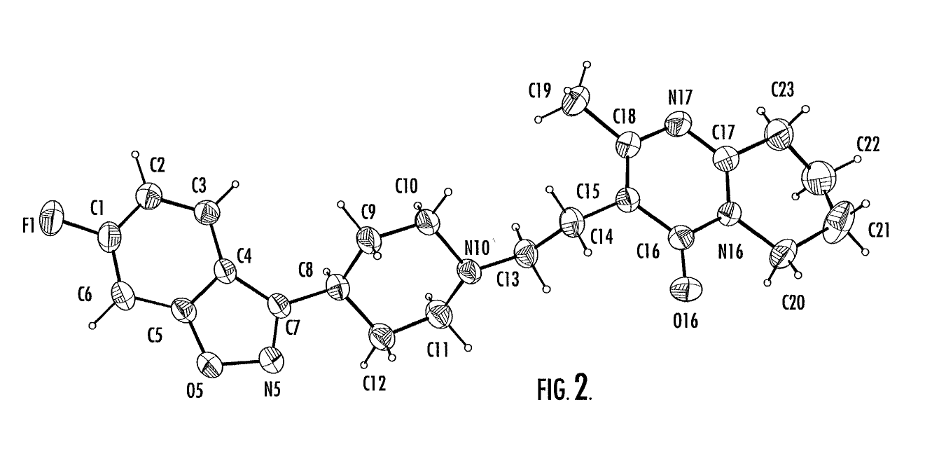

As seen in FIGS. 1 and 2, ORTEP drawings of the single crystal structures of Form A of risperidone and Form B of risperidone, respectively, show the different conformational orientations of the two risperidone molecules for Form A, thereby distinguishing these two forms of risperidone. The ORTEP drawings are generated from the Oak Ridge Thermal Ellipsoid Program developed by Oak Ridge National Laboratory in Oak Ridge, Tenn.

Form A risperidone was consistently prepared from the recrystallization of risperidone API, obtained from TEVA Pharmaceutical Industries Ltd, of Petah Tiqva, Israel (herein referred to as “TEVA Risperidone API”), as taught herein. It is estimated that the TEVA Risperidone API sample contained between 30% and 35% Form A risperidone and between 65% and 70% Form B risperidone. Form B risperidone was prepared as disclosed in U.S. Pat. No. 4,804,663 to Kennis et al., the disclosure of which is herein incorporated by reference, as well as other methods taught herein. Mixtures of Form A and Form B were prepared from recrystallization of the TEVA Risperidone API and Form B risperidone, as taught herein.

X-ray single crystal unit cell parameters for Form A of risperidone, Form B of risperidone and Reported Risperidone are compared in Table 1, below:

TABLE 1
X-Ray Single Crystal Unit Cell Parameters for Form A,
Form B, and Reported Risperidone
(numbers in the parenthesis are the estimated
error for each measurement)

| | Form A | Form B | Reported |
|---|---|---|---|
| Crystal Lattice | Triclinic | Monoclinic | Monoclinic |
| Space Group | P1 BAR | P2₁/n | P2₁/n |
| a | 9.9555(4) Å | 14.2326(3) Å | 14.24(1) Å |
| b | 11.0489(5) Å | 9.7636(2) Å | 9.767(7) Å |
| c | 20.3060(10) Å | 16.5911(5) Å | 16.59(1) Å |
| α | 75.207(2)° | — | — |
| β | 79.542(2)° | 113.734(1)° | 113.74(6)° |
| γ | 81.416(4)° | — | — |
| V(Å³) | 2111.2(2) Å³ | 2110.5(2) Å³ | 2112(3) Å³ |
| Z | 4 | 4 | 4 |
| FW | 410.50 amu | 410.50 amu | 410.49 amu |
| Dcalc | 1.291 g cm⁻³ | 1.292 g cm⁻³ | 1.2912 g cm⁻³ |

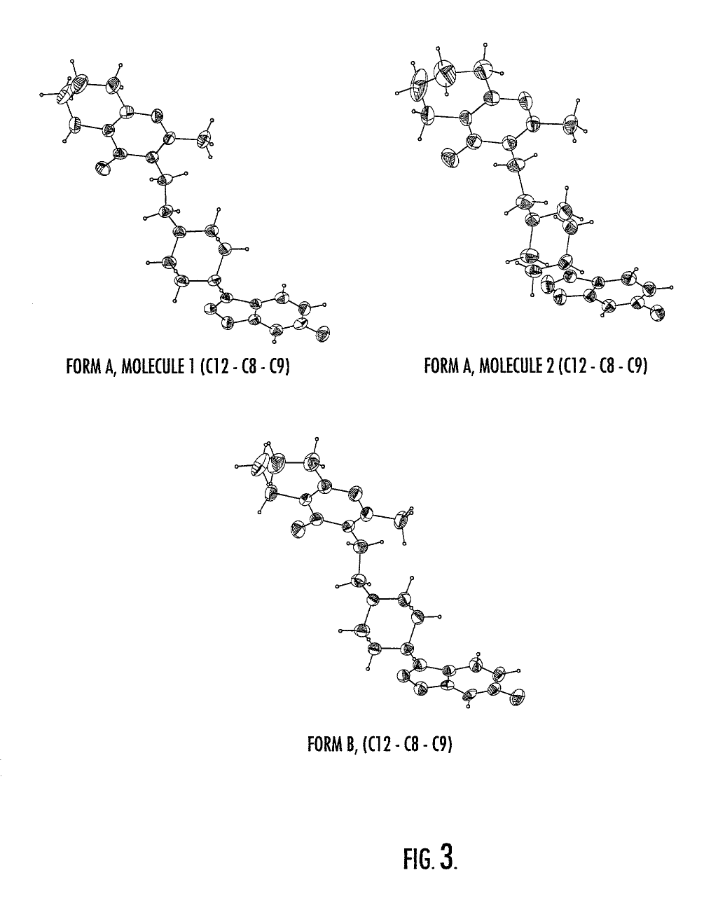

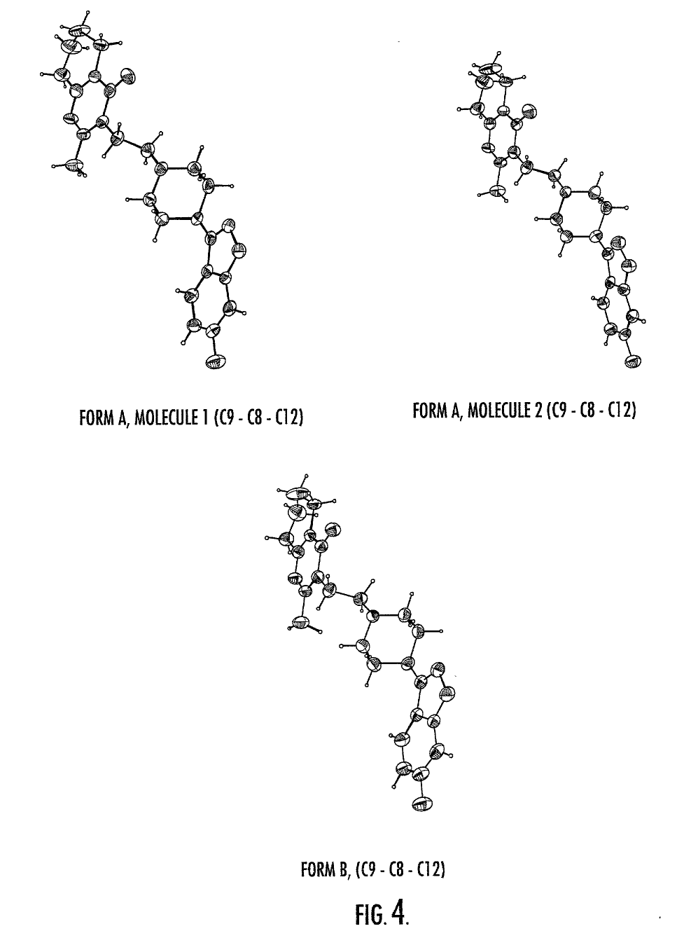

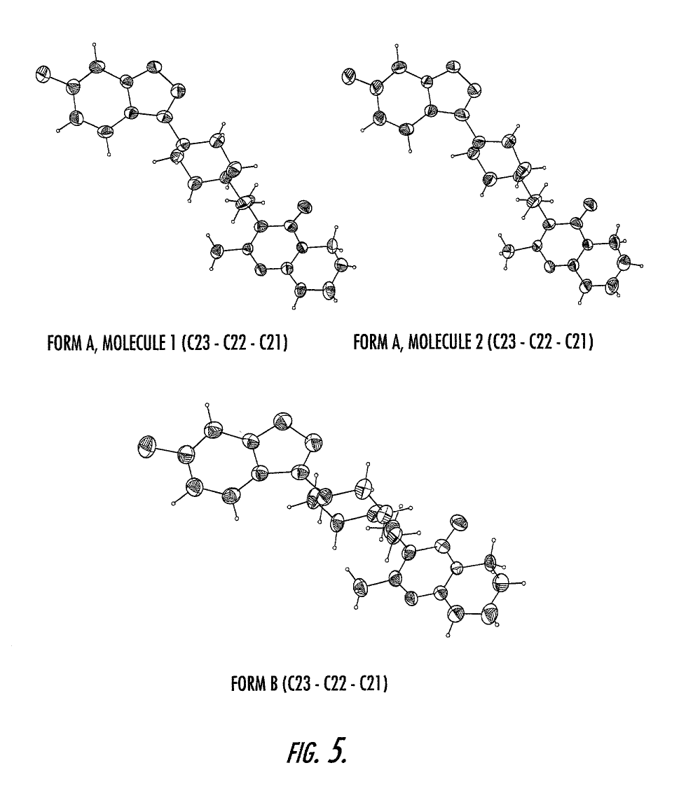

As seen in Table 1, Form B and Reported Risperidone are the same polymorphic form, and are distinct from Form A. The orientation of Form A and Form B is further shown, as a comparison, in FIGS. 3-5. As seen in these figures, Form A comprises two molecules per asymmetric unit, and Form B comprises one molecule per asymmetric unit. FIG. 3 shows an ORTEP drawing comparison of two unique molecules in the single crystal structure of Form A risperidone to Form B risperidone viewed along the C12-C8-C9 plane, FIG. 4 shows an ORTEP drawing comparison of two unique molecules in the single crystal structure of Form A risperidone to Form B risperidone viewed along the C9-C8-C12 plane, and FIG. 5 shows an ORTEP drawing comparison of two unique molecules in the single crystal structure of Form A risperidone to Form B risperidone viewed along the C23-C22-C21 plane;

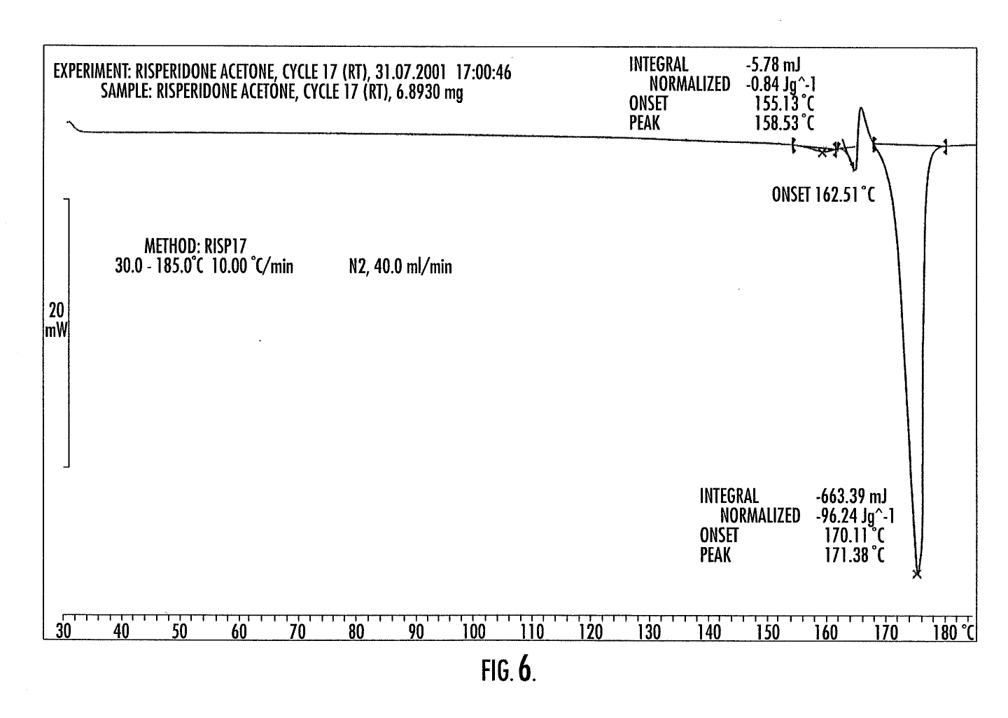

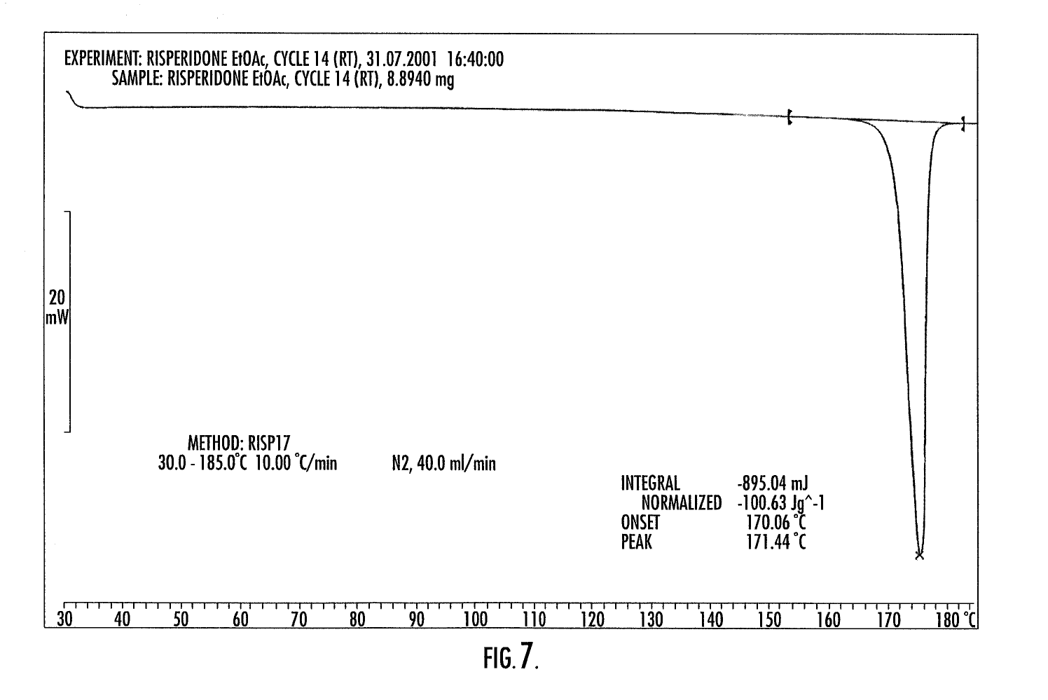

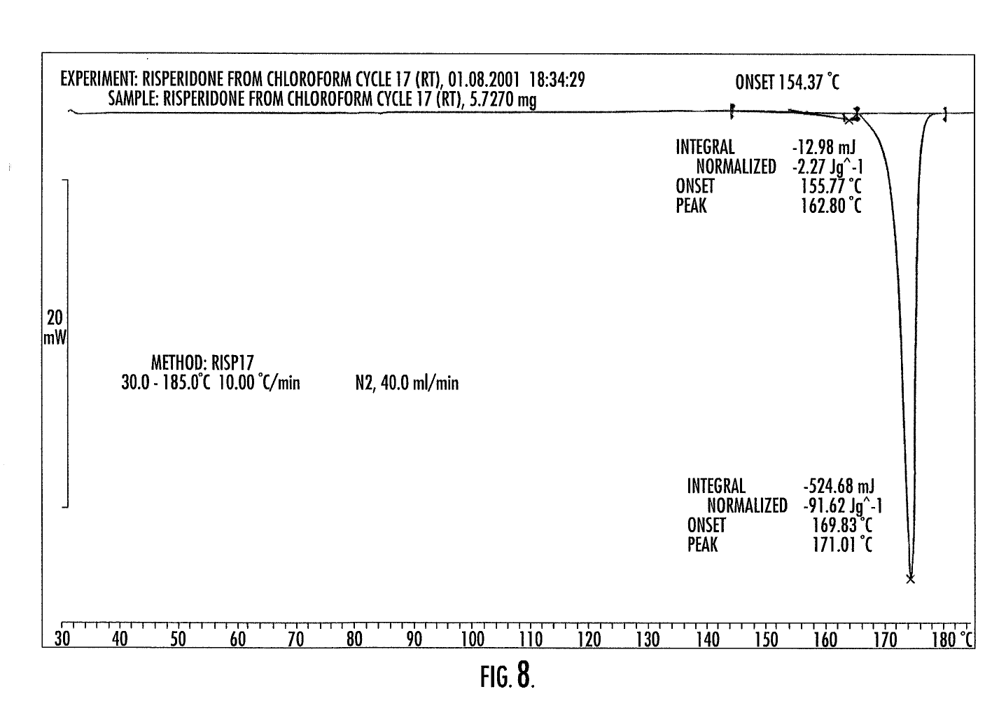

Furthermore, characterization of Form A of risperidone and Form B of risperidone was further completed using DSC thermograms, shown in FIGS. 6 and 7, respectively, with DSC thermograms for combinations of Form A and Form B shown in FIG. 8. DSC data were generated using a Mettler-Toledo DSC 821^{e} (Columbus, Ohio) with a Julabo FT900 intercooler chiller (Julabo Company; Allentown, Pa.). In general, samples were analyzed in a vented, sealed aluminum pan. Because the endothermic peak may vary depending upon the rate of heating and the calibration and precision of the instrument, with the amount of peak variation dependent upon the heating rate used, all thermograms included herein were run under the same, consistent conditions: heating at 10° C. per minute under a nitrogen purge at 40 mL per minute.

As seen in FIG. 6, the DSC thermogram for Form A gives an endothermic peak at about 164° C. (onset at about 155° C.), during melt Form A appears to undergo solid-state phase transformation to Form B. During continuous heating above 164° C., an exothermic peak appears between about 164° C. and 167° C., followed by a second endothermic peak of melt at about 171.5° C. (onset at about 170° C.). The DSC thermogram shown in FIG. 7 shows only a single endothermic peak for Form B at about 171.5° C. (onset at about 170° C.) which correlates with the second DSC endothermic peak observed in Form A. As previously noted, Form A undergoes a solid-state phase transformation to From B during heating.

The DSC thermogram in FIG. 8 shows a risperidone sample, with both Form A and Form B present, as determined by X-ray powder diffraction, discussed below. The DSC thermogram again shows conversion of Form A to Form B during heating, with Form A of risperidone appearing at about 162.5° C. (onset at about 155° C.) and Form B appearing at about 171° C. (onset at about 169.5° C.) during this heating cycle.

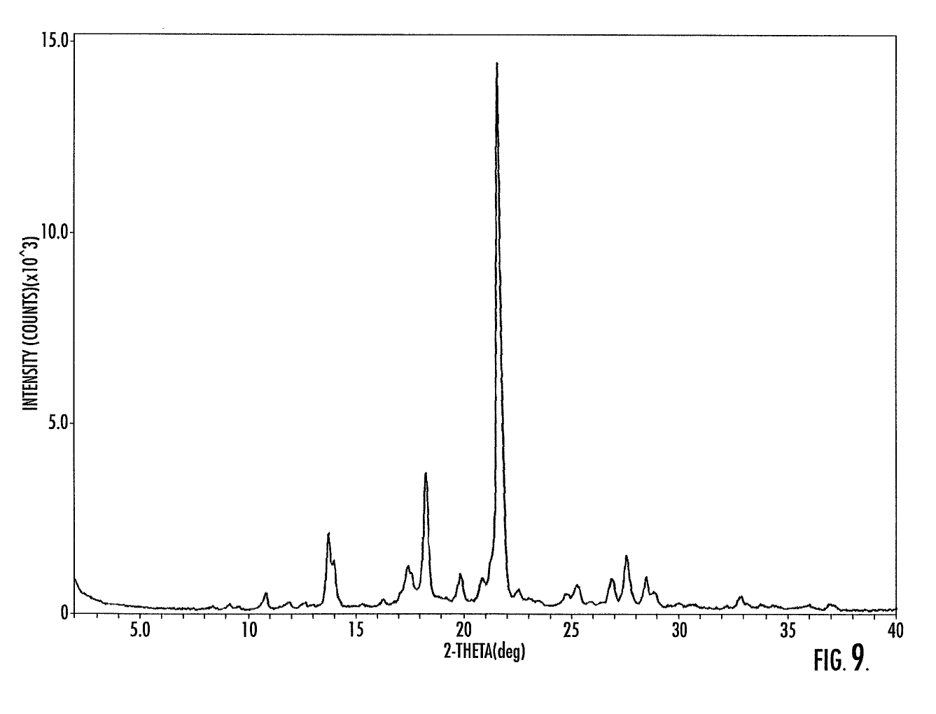

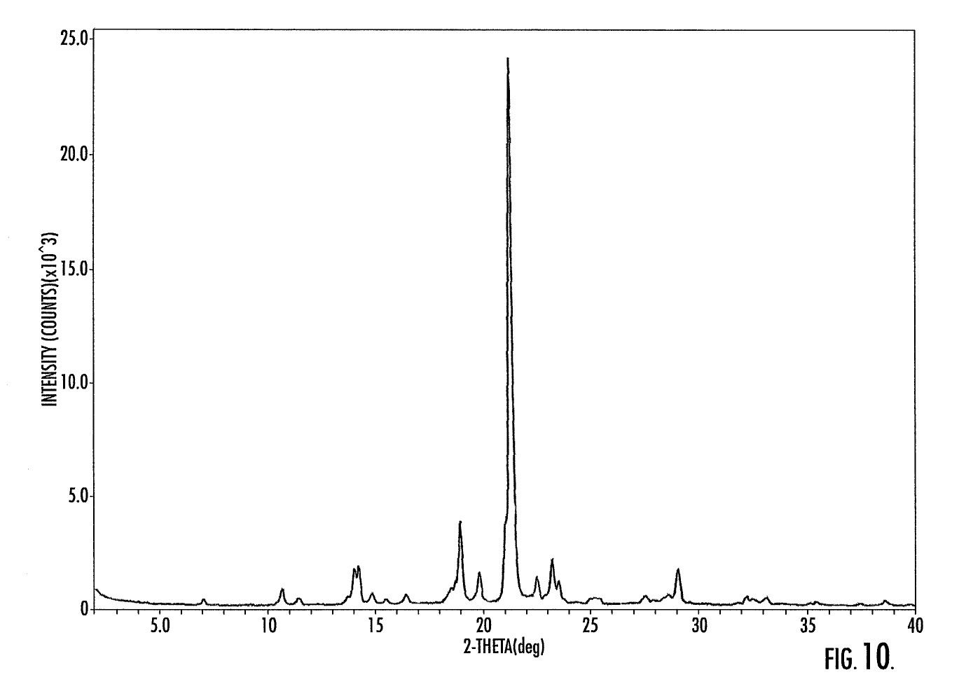

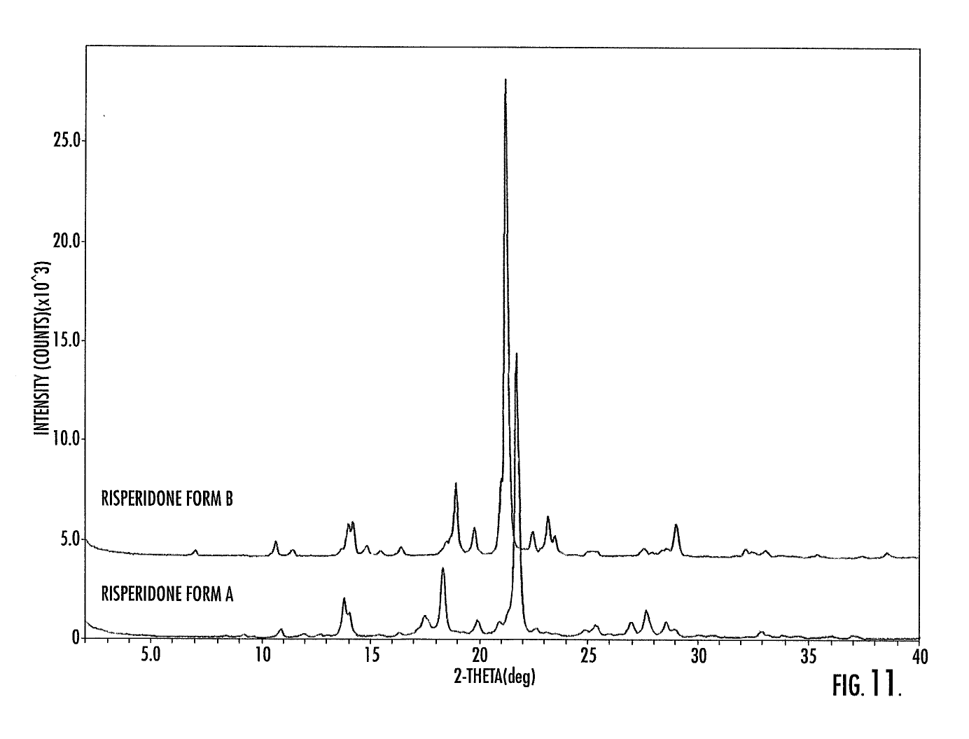

X-ray powder diffraction is another tool typically available for the characterization of mixtures of polymorphs and individual polymorphs of the same substance. X-ray powder diffraction was used to further identify and distinguish pure Form A. For additional confirmation of the presence of Form A of risperidone, X-ray powder diffraction and differential scanning calorimetry can be used together. In FIGS. 9 and 10, the XRD patterns for Form A (FIG. 9) and Form B (FIG. 10) are shown, with the XRD patterns for Form A and Form B overlayed for comparison in FIG. 11. As seen in FIG. 11, the XRD patterns of Form A and Form B of risperidone demonstrate distinct crystalline forms of the risperidone, showing pure Form A and pure Form B. XRD was performed using a Siemens D500 Diffractometer (Madison, Wis.). Samples were analyzed from 2-40° in 2 θ at 2.4° per minute using CuKα (50 kV, 30 mA) radiation on a zero-background sample plate.

Tabulations of the peak positions from the X-ray powder patterns for Form A and Form B are listed in Tables 2 and 3, below. It is well known by one skilled in the art that lot-to-lot variations of crystal shape and/or size, as well as variations among instruments and calibration of such instruments, can appear as preferred orientation in the X-ray powder diffraction patterns. This preferred orientation can be seen as variations in the relative intensities of the peaks, with variations in intensities of over 20%.

TABLE 2
X-Ray Powder Diffraction Peaks of Form A of Risperidone

| 2-Theta (degrees) | d(Å) | Intensity |
|---|---|---|
| 10.88 | 8.12 | very weak |
| 11.94 | 7.41 | Very weak |
| 13.80 | 6.41 | moderate |
| 14.03 | 6.31 | moderate |
| 16.30 | 5.43 | very weak |
| 17.50 | 5.06 | weak |
| 18.34 | 4.83 | Moderate |
| 19.91 | 4.46 | weak |
| 20.95 | 4.24 | weak |
| 21.34 | 4.16 | weak |
| 21.79 | 4.07 | strong |
| 22.63 | 3.93 | very weak |
| 24.85 | 3.58 | very weak |
| 25.30 | 3.52 | very weak |
| 26.91 | 3.31 | weak |
| 27.61 | 3.23 | weak |
| 28.55 | 3.12 | weak |
| 28.90 | 3.09 | very weak |
| 32.93 | 2.72 | very weak |

TABLE 3
X-Ray Powder Diffraction
Significant Peaks of Form B of Risperidone

| 2-Theta (degrees) | d(Å) | Intensity |
|---|---|---|
| 7.03 | 12.56 | very weak |
| 10.65 | 8.30 | very weak |
| 11.41 | 7.75 | very weak |
| 13.71 | 6.45 | very weak |
| 14.01 | 6.32 | Weak |
| 14.20 | 6.23 | Weak |
| 14.84 | 5.96 | very weak |
| 15.49 | 5.72 | very weak |
| 16.40 | 5.40 | very weak |
| 18.54 | 4.78 | very weak |
| 18.95 | 4.68 | Moderate |
| 19.80 | 4.48 | Weak |
| 21.05 | 4.22 | Moderate |
| 21.31 | 4.17 | Strong |
| 22.48 | 3.95 | Weak |
| 23.20 | 3.83 | Moderate |
| 23.49 | 3.78 | Weak |
| 25.16 | 3.54 | very weak |
| 25.39 | 3.51 | very weak |
| 27.51 | 3.24 | very weak |
| 28.56 | 3.12 | very weak |
| 29.01 | 3.08 | Weak |
| 32.46 | 2.76 | very weak |
| 38.55 | 2.33 | very weak |

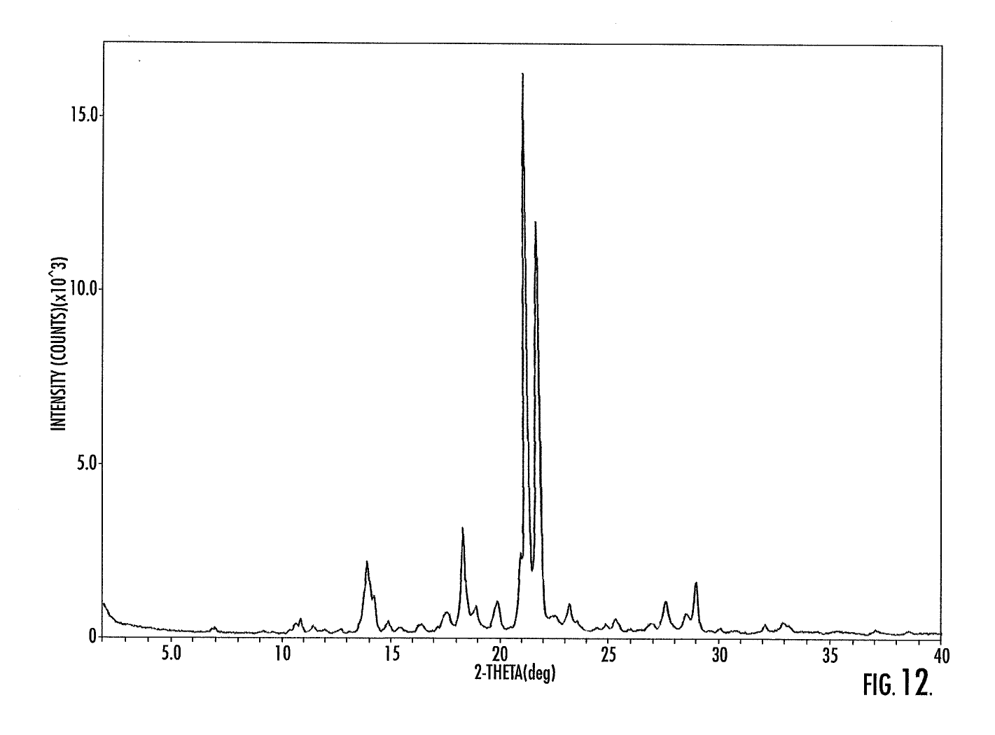

The XRD peaks shown in Table 2, demonstrated that characterization peaks of Form A are typically located at two-theta (2θ) angles of about 10.9, 11.9, 13.8, 14.0, 16.3, 17.5, 18.3, 19.9, 21.3, 21.8, and 26.9°, with d(Å) values of about 8.12, 7.41, 6.41, 6.31, 5.43, 5.06, 4.83, 4.46, 4.16, 4.07, and 3.31, respectively. For Form B, characterization XRD peaks (shown in Table 3) are at two-theta (2θ) angles of about 7.0, 10.7, 11.4, 14.0, 14.2, 14.8, 16.4, 19.0, 19.8, 21.1, 21.3, 22.5, 23.2, 23.5, 29.0, and 38.6°, with d(Å) values of about 12.56, 8.30, 7.75, 6.32, 6.23, 5.96, 5.40, 4.68, 4.48, 4.22, 4.17, 3.95, 3.83, 3.78, 3.08, and 2.33, respectively. The XRD pattern for the mixture of Form A of risperidone Form B of risperidone is shown in FIG. 12.

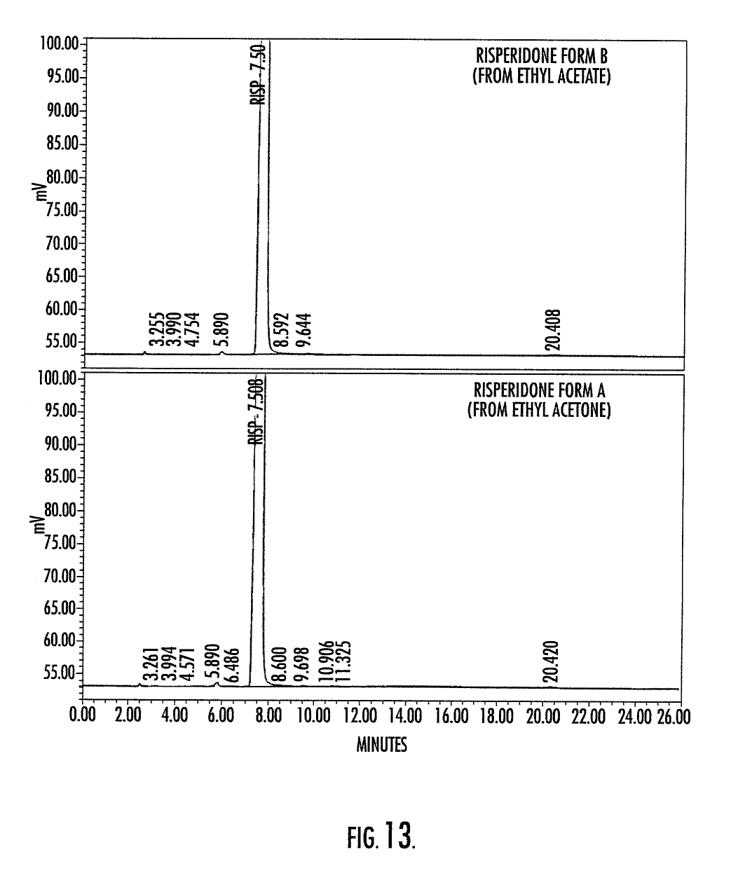

The HPLC Chromatogram of Form A was overlayed with the chromatogram of a polymorphic Form B sample, as shown in FIG. 13. This figure shows that no degradation occurred during solvent recrystallization of the risperidone sample, with a total amount of impurities of less than about 0.2% in each polymorphic form.

Accordingly, Form A and Form B polymorphic forms of risperidone have been characterized as distinct from each other. XRC, DSC, XRD, and HPLC confirm the existence and/or purity of the novel Form A of risperidone, as distinct from Form B of risperidone.

In preparing Form A of risperidone, recrystallization of risperidone was performed. Risperidone API is recrystallized (or crystallized in situ, as the case may be) into Form A by dissolving such API in a suitable solvent in excess. Suitable solvents are those which are capable of dissolving risperidone so that a solution is formed, and include solvents across various classes including, for example, protic, aprotic, polar, and non-polar solvents. The resulting solution is filtered and permitted to recrystallize, most preferably at a fixed temperature, by evaporation. The temperature used for the evaporation step should be held constant at a temperature which permits the recrystallization of the starting material to form Form A. A temperature range from about 0° C. to about 60° C. is preferred, while a temperature range from about 15° C. to about 40° C. is more preferred, and about ambient temperature (from about 20° C. to about 25° C.) is most preferred. This method has provided pure and substantially pure Form A of risperidone depending upon whether this recrystallization process is allowed to run to completion. Solvents systems having nitriles, ketones or alcohols tend to form pure or substantially pure Form A risperidone. Aliphatic alcohol-based solvents are preferred, as are solvent systems containing low molecular weight alcohols. More preferred are solvent systems containing acetonitrile, acetone, methanol or ethanol, with acetone most preferred.

Preferably, Form A is produced in a pure form (devoid of detectable amounts of other polymorphic forms of risperidone as determined by X-ray powder diffraction or other appropriate methods of characterization), having negligible amounts of other detectable polymorphic forms of risperidone, or in substantially pure form.

Preparation of Form B of risperidone was accomplished by recrystallization of risperidone, generally using low vapor pressure solvent systems, such as chlorinated solvent systems or ester solvent systems, particularly ethyl acetate. Solvents for the preparation of Form B include, for example, ethyl acetate, dichloromethane, dimethylfarmamide with isopropanol, dimethylformamide, and ethyl acetate.

Preparation of Form A and Form B mixtures of risperidone may be accomplished from crystallization of risperidone from the above-identified solvent systems, under similar conditions, by imparting a physical disturbance or nucleation of the crystallizing risperidone. Physical disturbances are, for example, an external force imparted to the recrystallizing risperidone, such as vibrations, probing, seeding, dust particles or air currents.

Preparation of Form A, individually and with Form B, from crystallization, or recrystallization, from a solvent may vary with physical handling or environmental factors, such as cooling rates, physical disturbance, nucleation, evaporation rates, and other such factors which are controllable for consistent replication of the desired crystalline form of risperidone, as understood by one skilled in the art.

Mixtures of Form A and Form B also may be created by blending or mixing compositions of Form A and Form B together, e.g., from samples prepared individually for Form A and Form B as described herein, in appropriate amounts.

The present invention also provides pharmaceutical formulations comprising Form A risperidone, in pure or other form, either as the sole active ingredient or in combination with other active ingredients including, for example, other polymorphic forms of risperidone (e.g., Form B) or other pharmaceutically active agents, and at least one pharmaceutically acceptable carrier, diluent, and/or excipient. Combinations of more than one polymorphic form of risperidone are prepared via the described crystallization procedures or, for more precise combinations, via blending of pure or known polymorphic forms to desired ratios. Preferably the novel crystalline form of risperidone, Form A, is in pure form.

For the most effective administration of the polymorphic forms of the present invention, it is preferred to prepare a pharmaceutical formulation preferably in unit dose form, comprising one or more of the active ingredients of the present invention and one or more pharmaceutically acceptable carrier, diluent, or excipient.

As used herein, the term “active ingredient” refers to Form A, individually and in combination among polymorphic forms of the present invention or other risperidone polymorphic forms. More preferably polymorphic Form A of the present invention is used in pure form in the pharmaceutical formulations of the present invention.

Preferred pharmaceutical formulations may include, without being limited by the teachings as set forth herein, a solid dosage form of Form A with and without Form B, with at least one pharmaceutically acceptable excipient, diluted by an excipient or enclosed within such a carrier that can be in the form of a capsule, sachet, tablet, buccal, lozenge, paper, or other container. Additionally, such pharmaceutical formulation may include a liquid formulation prepared from Form A risperidone API of the present invention in combination with at least one pharmaceutically acceptable excipient, diluted by an excipient or enclosed within an appropriate carrier. When the excipient serves as a diluent, it may be a solid, semi-solid, or liquid material which acts as a vehicle, carrier, or medium for the active ingredient(s). Thus, the formulations can be in the form of tablets, pills, powders, elixirs, suspensions, emulsions, solutions, syrups, capsules (such as, for example, soft and hard gelatin capsules), suppositories, sterile injectable solutions, and sterile packaged powders.

Examples of suitable excipients include, but are not limited to, starches, gum arabic, calcium silicate, microcrystalline cellulose, polyvinylpyrrolidone, cellulose, water, syrup, and methyl cellulose. The formulations can additionally include lubricating agents such as, for example, talc, magnesium stearate and mineral oil; wetting agents; emulsifying and suspending agents; preserving agents such as methyl- and propyl-hydroxybenzoates; sweetening agents; or flavoring agents. Polyols, buffers, and inert fillers may also be used. Examples of polyols include, but are not limited to: mannitol, sorbitol, xylitol, sucrose, maltose, glucose, lactose, dextrose, and the like. Suitable buffers encompass, but are not limited to, phosphate, citrate, tartrate, succinate, and the like. Other inert fillers which may be used encompass those which are known in the art and are useful in the manufacture of various dosage forms. If desired, the solid pharmaceutical compositions may include other components such as bulking agents and/or granulating agents, and the like. The compositions of the invention can be formulated so as to provide quick, sustained, controlled, or delayed release of the active ingredient after administration to the patient by employing procedures well known in the art.

In certain embodiments of the present invention, the active ingredient(s) may be made into the form of dosage units for oral administration. The active ingredient(s) may be mixed with a solid, pulverant carrier such as, for example, lactose, saccharose, sorbitol, mannitol, starch, amylopectin, cellulose derivatives or gelatin, as well as with an antifriction agent such as for example, magnesium stearate, calcium stearate, and polyethylene glycol waxes. The mixture is then pressed into tablets or filled into capsules. If coated tablets, capsules, or pulvules are desired, such tablets, capsules, or pulvules may be coated with a concentrated solution of sugar, which may contain gum arabic, gelatin, talc, titanium dioxide, or with a lacquer dissolved in the volatile organic solvent or mixture of solvents. To this coating, various dyes may be added in order to distinguish among tablets with different active compounds or with different amounts of the active compound present.

Soft gelatin capsules may be prepared in which capsules contain a mixture of the active ingredient(s) and vegetable oil or non-aqueous, water miscible materials such as, for example, polyethylene glycol and the like. Hard gelatin capsules may contain granules or powder of the active ingredient in combination with a solid, pulverulent carrier, such as, for example, lactose, saccharose, sorbitol, mannitol, potato starch, corn starch, amylopectin, cellulose derivatives, or gelatin.

Tablets for oral use are typically prepared in the following manner, although other techniques may be employed. The solid substances are gently ground or sieved to a desired particle size, and a binding agent is homogenized and suspended in a suitable solvent. The active ingredient(s) and auxiliary agents are mixed with the binding agent solution. The resulting mixture is moistened to form a uniform suspension. The moistening typically causes the particles to aggregate slightly, and the resulting mass is gently pressed through a stainless steel sieve having a desired size. The layers of the mixture are then dried in controlled drying units for a pre-determined length of time to achieve a desired particle size and consistency. The granules of the dried mixture are gently sieved to remove any powder. To this mixture, disintegrating, anti-friction, and anti-adhesive agents are added. Finally, the mixture is pressed into tablets using a machine with the appropriate punches and dies to obtain the desired tablet size.

Liquid preparations for oral administration are prepared in the form of solutions, syrups, or suspensions with the latter two forms containing, for example, active ingredient(s), sugar, and a mixture of ethanol, water, glycerol, and propylene glycol. If desired, such liquid preparations contain coloring agents, flavoring agents, and saccharin. Thickening agents such as carboxymethylcellulose may also be used.

As such, the pharmaceutical formulations of the present invention are preferably prepared in a unit dosage form, each dosage unit containing from about 0.1 mg to about 100 mg, preferably from about 0.1 mg to about 10 mg, and more preferably from about 0.25 mg to about 8 mg of the risperidone active ingredient(s), with representative dosage units of 0.25 mg, 0.5 mg, 1 mg, 2 mg, 3 mg, 4 mg and 8 mg. Other pharmaceutically active agents can also be added to the pharmaceutical formulations of the present invention at therapeutically effective dosages. In liquid form, unit doses contain from about 0.01 mg to about 100 mg, preferably from about 0.1 mg to about 10 mg, and more preferably from about 0.25 mg to about 8 mg of such risperidone active ingredient(s).

The term “unit dosage form” refers to physically discrete units suitable as unitary dosages for human subjects/patients or other mammals, each unit containing a predetermined quantity of active ingredient calculated to produce the desired therapeutic effect, in association with preferably, at least one pharmaceutically acceptable carrier, diluent, or excipient.

The invention also provides methods of treating a subject (e.g., mammal, particularly humans) comprising administering to a subject in need of such treatment a therapeutically effective amount of at least one active ingredient, formulation thereof, or unit dose forms thereof, each as described herein. The active ingredient(s) are used as an antipsychotic, in the management of manifestations of psychotic disorders.

As used herein, the term “treatment”, or a derivative thereof, contemplates partial or complete mitigation of psychotic disorders such as, for example, conceptual disorganization, hallucinatory behavior, suspiciousness, and unusual thought content, when an active ingredient of the present invention is administered prophylactically or following the onset of the disease state for which such active ingredient of the present invention is administered. For the purposes of the present invention, “prophylaxis” refers to administration of the active ingredient(s) to a subject to protect the subject from any of the disorders set forth herein, as well as others.

The typical active daily dose of the risperidone active ingredient(s) will depend on various factors such as, for example, the individual requirement of each patient, the route of administration, and the disease state. An attending physician may adjust the dosage rate based on these and other criteria if he or she so desires. A suitable daily dosage, typically administered BID in equally divided doses, is from about 0.25 mg to about 4 mg, preferably from about 0.5 mg to about 3 mg, and more preferably from about 1 mg to about 2 mg. A preferred range is from about 0.25 mg to about 4 mg total daily dose. It should be appreciated that daily doses other than those described above may be administered to a subject, as appreciated by an attending physician. Once a day dosages may be administered in appropriate amounts as prescribed and needed.

The following examples are for illustrative purposes only and are not intended to limit the scope of the claimed invention. Characterization of each of Example 1-12 was done by XRD and DSC, with the results of the characterization shown in Table 4.

### EXAMPLE 1

### Preparation of Pure Form A of Risperidone

To a flask containing 60 mL of acetone was added approximately 200 mg of risperidone API. The mixture was stirred sufficiently to dissolve the risperidone in the acetone while heating to about 45° C. The final solution was filtered using a 0.45 μm PTFE filter and allowed to crystallize by evaporation of the solvent at ambient conditions, protected from dust and vibration. After evaporation of the solvent, risperidone crystals obtained were further dried under vacuum at room temperature.

### EXAMPLE 2

### Preparation of Pure Form A of Risperidone

To a flask containing 350 mL of acetone was added approximately 1 g of risperidone API. The mixture was stirred sufficiently to dissolve the risperidone in the acetone at room temperature. The final solution was filtered using a 0.45 μm PTFE filter and allowed to crystallize by evaporation of the solvent at ambient conditions, protected from dust and vibration. After evaporation of the solvent, risperidone crystals obtained were further dried under vacuum at room temperature.

### EXAMPLE 3

### Preparation of Pure Form A of Risperidone

To a flask containing 30 mL of methanol was added approximately 200 mg of risperidone API. The mixture was stirred sufficiently to dissolve the risperidone in the methanol at room temperature. The final solution was filtered using a 0.45 μm PTFE filter and allowed to crystallize by evaporation of the solvent at ambient conditions, protected from dust and vibration. After evaporation of the solvent, risperidone crystals obtained were further dried under vacuum at room temperature.

### EXAMPLE 4

### Preparation of Pure Form A of Risperidone

To a flask containing 60 mL of ethanol was added approximately 200 mg of risperidone API. The mixture was stirred sufficiently to dissolve the risperidone in the ethanol while heating to about 45° C. The final solution was filtered using a 0.45 μm PTFE filter and allowed to crystallize by evaporation of the solvent at ambient conditions, protected from dust and vibration. After evaporation of the solvent, risperidone crystals obtained were further dried under vacuum at room temperature.

### EXAMPLE 5

### Preparation of Pure Form A of Risperidone

To a flask containing 160 mL of acetonitrile was added approximately 200 mg of risperidone API. The mixture was stirred sufficiently to dissolve the risperidone in the acetonitrile while heating to about 45° C. The final solution was filtered using a 0.45 μm PTFE filter and allowed to crystallize by evaporation of the solvent at ambient conditions, protected from dust and vibration. After evaporation of the solvent, risperidone crystals obtained were further dried under vacuum at room temperature.

### EXAMPLE 6

### Preparation of Pure Form B of Risperidone

To a flask containing 100 mL of ethyl acetate was added approximately 200 mg of risperidone API. The mixture was stirred sufficiently to dissolve the risperidone in the ethyl acetate while heating to about 45° C. The final solution was filtered using a 0.45 μm PTFE filter and allowed to crystallize by evaporation of the solvent at ambient conditions, protected from dust and vibration. After evaporation of the solvent, risperidone crystals obtained were further dried under vacuum at room temperature.

### EXAMPLE 7

### Preparation of Pure Form B of Risperidone

To a flask containing 300 mL of ethyl acetate was added approximately 1.1 g of risperidone API. The mixture was stirred sufficiently to dissolve the risperidone in the ethyl acetate at room temperature. The final solution was filtered using a 0.45 μm PTFE filter and allowed to crystallize by evaporation of the solvent at ambient conditions, protected from dust and vibration. After evaporation of the solvent, risperidone crystals obtained were further dried under vacuum at room temperature.

### EXAMPLE 8

### Preparation of Pure Form B of Risperidone

To a flask containing 30 mL of dichloromethane was added approximately 200 mg of risperidone API. The mixture was stirred sufficiently to dissolve the risperidone in the dichloromethane at room temperature. The final solution was filtered using a 0.45 μm PTFE filter and allowed to crystallize by evaporation of the solvent at ambient conditions, protected from dust and vibration. After evaporation of the solvent, risperidone crystals obtained were further dried under vacuum at room temperature.

### EXAMPLE 9

### Preparation of Pure Form B of Risperidone

To a flask containing 30 mL of dimethylformamide (DMF) was added approximately 200 mg of risperidone API. The mixture was stirred sufficiently to dissolve the risperidone in the dimethylformamide (DMF) at room temperature. The solution was filtered using a 0.45 μm PTFE filter and aliquot of 15 mL dimethylformamide was mixed with 60 mL of isopropanol (IPA). The DMF/IPA solution was allowed to evaporate at ambient conditions under a very gentle stream of nitrogen gas. After crystallization had taken place, the leftover solvent was removed and the crystals were dried by vacuum filtration.

### EXAMPLE 10

### Preparation of Mixtures of Form A and Form B of Risperidone

To a flask containing 30 mL of dimethylformamide (DMF) was added approximately 200 mg of risperidone API. The mixture was stirred sufficiently to dissolve the risperidone in the DMF using stirring at room temperature. The solution was filtered using a 0.45 μm PTFE filter and aliquot of 15 mL DMF was allowed to evaporate at ambient conditions under a stream of nitrogen gas. After crystallization had taken place, the leftover solvent was removed and the crystals were dried by vacuum filtration.

### EXAMPLE 11

### Preparation of Mixtures of Form A and Form B of Risperidone

To a flask containing 30 mL of acetone was added approximately 100 mg of risperidone API. The mixture was stirred sufficiently to dissolve the risperidone at room temperature. The solution was filtered using a 0.45 μm PTFE filter and allowed to evaporate at ambient temperature under a stream of nitrogen gas.

### EXAMPLE 12

### Preparation of Mixtures of Form A and Form B of Risperidone

To a flask containing 35 mL of ethyl acetate was added approximately 100 mg of risperidone API. The mixture was stirred sufficiently to dissolve the risperidone in the ethyl acetate at room temperature. The solution was filtered using a 0.45 μm PTFE filter and allowed to evaporate at ambient temperature under a stream of nitrogen gas.

TABLE 5

| Example | Solvent | Form |
|---|---|---|
| Example 1 | Acetone | A |
| Example 2 | Acetone | A |
| Example 3 | Methanol | A |
| Example 4 | Ethanol | A |
| Example 5 | Acetonitrile | A |
| Example 6 | Ethyl Acetate | B |
| Example 7 | Ethyl Acetate | B |
| Example 8 | Dichloromethane | B |
| Example 9 | Dimethylformamide (DMF) and Isopropanol | B |
| | (IPA) | |
| Example 10 | Dimethylformamide (DMF) | A & B |
| Example 11 | Acetone (under N₂ stream) | A & B |
| Example 12 | Ethyl Acetate (under N₂ stream) | A & B |

### EXAMPLE 13

### Preparation of Mixtures of Form A and Form B of Risperidone

A sample of approximately 5 mg of Form B of risperidone was placed in a vented, sealed aluminum holder and placed in a DSC furnace. Under a nitrogen purge of 40 mL per minute, the sample was heated from a temperature of about 30° C. to about 180° C. (past the melting point of Form B) at a heating rate of about 10° C. per minute. The molten risperidone was cooled within the furnace to about 0° C. at a cooling rate of about 10° C. per minute. The cooled risperidone was then reheated in an undisturbed state in the DSC oven at a rate of 10° C. per minute to a final temperature above 180° C. The sample showed an endothermic peak for Form A of risperidone at about 165° C. (onset at about 135° C.) with a second endothermic peak at about 171° C. (onset at about 169° C.) which related to Form B of risperidone. XRD confirmed mixtures of Form A and Form B, and the DSC thermogram showed the presence of Form A in the sample.

### Formulation 1

Hard gelatin 1 mg capsules are prepared using the following ingredients:

| | Quantity (mg per capsule) |
|---|---|
| active ingredient(s) | 1 mg |
| lactose monohydrate | 165 mg |
| polyvinylpyrrolidone (PVP) | 10 mg |
| croscarmellose sodium | 15 mg |
| magnesium stearate | 10 mg |
| Total | 201 mg |

The above ingredients are mixed and filled into hard gelatin Capsules in about 201 mg quantities.

### Formulation 2

A 4 mg tablet is prepared using the ingredients below:

| | Quantity (mg per tablet) |
|---|---|
| active ingredient(s) | 4 mg |
| cellulose, microcrystalline | 400 mg |
| silicon dioxide, fumed | 10 mg |
| stearic acid | 5 mg |
| Total | 419 mg |

The components are blended and compressed to form tablets each weighing about 419 mg.

### Formulation 3

Tablets each containing 0.25 mg of active ingredient are made as follows:

| | |
|---|---|
| active ingredient | 0.25 mg |
| anhydrous lactose | 45 mg |
| cellulose, microcrystalline | 35 mg |
| polyvinylpyrrolidone (PVP) | 4 mg |
| sodium carboxymethyl starch | 4 mg |
| magnesium stearate | 0.75 mg |
| talc | 1 mg |
| Total | 90 mg |

The active ingredient, starch and cellulose are passed through a No. 45 mesh U.S. sieve and mixed thoroughly. The solution of polyvinylpyrrolidone is mixed with the resultant powders which are then passed through a No. 14 mesh U.S. sieve. The granules so produced are dried at 50° C. and passed through a No. 18 mesh U.S. sieve. The sodium carboxymethyl starch, magnesium stearate and talc, previously passed through a No. 60 mesh U.S. sieve, are then added to the granules which, after mixing, are compressed on a tablet machine to yield tablets each weighing about 90 mg.

### Formulation 4

Capsules each containing 0.5 mg of active ingredient are made as follows:

| | |
|---|---|
| active ingredient | 0.5 mg |
| starch | 59 mg |
| cellulose, microcrystalline | 59 mg |
| magnesium stearate | 1.5 mg |
| Total | 120 mg |

The active ingredient, cellulose, starch and magnesium stearate are blended, passed through a No. 45 mesh U.S. sieve, and filled into hard gelatin capsules in about 120 mg quantities.

What is claimed is:

1. A process for preparing Form A of risperidone, comprising the steps of
dissolving a risperidone sample in a suitable solvent; and,
crystallizing the risperidone from the solvent in a manner effective to form Form A of risperidone.

2. The process of claim 1, wherein the suitable solvent is selected from the group consisting of nitriles, ketones, and alcohol based solvents.

3. The process of claim 2, wherein the suitable solvent is selected from the group consisting of methanol, ethanol, acetone and acetonitrile.

4. The process of claim 3, wherein the suitable solvent is selected from the group consisting of methanol and ethanol.

5. The process of claim 3, wherein the suitable solvent is selected from the group consisting of acetone and acetonitrile.

6. Form A of risperidone prepared by the process of claim 1.

7. A process for preparing a mixture of two or more polymorphs of risperidone in a single mixture, comprising the steps of:
dissolving a risperidone sample in a suitable solvent; and
crystallizing the risperidone from the solvent in under physical disturbance wherein a mixture of Form A and at least a second polymorphic form of risperidone is formed.

8. The process of claim 7, wherein the physical disturbance comprises a stream of inert gas.

9. The process of claim 8, wherein the inert gas comprises nitrogen.

10. The process of claim 7, wherein the suitable solvent is selected from the group consisting of dimethylformamide and isopropanol, dimethylformamide, acetone and ethyl acetate.

11. A process for preparing a mixture of two or more polymorphic forms of risperidone in a single mixture, comprising the steps of:
heating a risperidone to a temperature of from about 172° C. or more; and
cooling the heated risperidone to a crystallization temperature of risperidone, wherein the risperidone crystallizes in two or more crystalline forms.

12. The process of claim 11, wherein at least one of the two or more polymorphs forms of risperidone comprises Form A of risperidone.

13. A process for preparing a composition of two or more polymorphs of risperidone in a single mixture, comprising the steps of:
selecting at least two different polymorphs of risperidone; and,
blending the two or more polymorphs of risperidone into a single mixture.

14. The process of claim 13, wherein the mixture is homogeneous.

15. The process of claim 13, wherein at least one of the two or more polymorphic forms of risperidone comprises form A of risperidone.
